(19) United States
(12) Patent Application Publication
(10) Pub. No.: US 2005/0100578 A1
(43) Pub. Date: May 12, 2005

(54) Bone and tissue scaffolding and method for producing same

(76) Inventors: Steven R. Schmid, Lakeville, IN (US); Glen L. Niebur, South Bend, IN (US); Ryan K. Roeder, Granger, IN (US)

Correspondence Address: JAGTIANI + GUTTAG, 10363-A DEMOCRACY LANE, FAIRFAX, VA, 22030, US

(21) Appl. No.: 10/980,425

(22) Filed: Nov. 4, 2004

Related U.S. Application Data
(60) Provisional application No. 60/517,408, filed on Nov. 6, 2003.

(51) Int. Cl. A61F 2/28; A61F 2/00

(52) U.S. Cl. 424/423; 623/16.11; 424/93.7

(57) ABSTRACT

The present invention provides a bone in-growth and on-growth material and method for making a material by bonding porous sheets together. The porosity is controllable from zero porosity to essentially a fully porous material.

### CROSS-REFERENCE TO RELATED APPLICATIONS

This application makes reference to co-pending U.S. Provisional Patent Application No. 60/517,408, entitled “Bone and Tissue Scaffolding and Method for Producing Same,” filed Nov. 6, 2003, the entire contents and disclosure of which is hereby incorporated by reference.

### BACKGROUND OF THE INVENTION

1. Field of the Invention

The present invention relates generally to orthopedic materials, and more particularly to a bone in-growth and on-growth material and soft tissue scaffolding with exceptional characteristics that may be manufactured for a reasonable cost.

2. Related Art

Materials with high porosity and possessing a controlled microstructure are of interest to implant manufacturers, particularly orthopedic implant manufacturers. Bone in-growth is known to preferentially occur in highly porous, open cell structures in which the cell size is roughly the same as that of trabecular bone (approximately 0.25-0.5 mm), with struts roughly 100 μm (0.1 mm) in diameter. For the orthopedic market, bone in-growth and on-growth options currently include the following: (a) DePuy Inc. sinters metal beads to implant surfaces, leading to a microstructure that is controlled and of a suitable pore size for bone in-growth, but with a lower than optimum porosity for bone in-growth; (b) Zimmer Inc. uses fiber metal pads produced by diffusion bonding loose fibers, wherein the pads are then diffusion bonded to implants or insert injection molded in composite structures, which also have lower than optimum density for bone in-growth; (c) Biomet Inc. uses a plasma sprayed surface that results in a roughened surface that produces on-growth, but does not produce bone in-growth; and (d) Implex Corporation produces HEDROCEL (also known as trabecular metal), using a chemical vapor deposition process to produce a tantalum-coated carbon microstructure that has also been called a metal foam. Research has suggested that HEDROCEL (trabecular metal) leads to high quality bone in-growth, see Bobyn, J. D., 1999, “Fixation and Bearing Surfaces for the Next Millenium”, Orthopedics, v. 22, pp. 810-822; Bobyn, J. D., et al., 1999, “Characteristics of Bone Ingrowth and Interface Mechanics of a New Porous Tantalum Biomaterial”, J. Bone and Joint Surgery, v. 81(5), pp. 907-914; and Bobyn, J. D., et al., 1999, “Tissue Response to Porous Tantalum Acetabular Cups”, J. Arthroplasty, v. 14, pp. 347-354, the entire contents and disclosures of which are hereby incorporated by reference. Trabecular metal has the advantages of high porosity, an open-cell structure and a cell size that is conducive to bone in-growth. However, trabecular metal has a chemistry and coating thickness that are difficult to control. Trabecular metal is very expensive, due to material and process costs and long processing times, primarily associated with chemical vapor deposition (CVD). Furthermore, CVD requires the use of very toxic chemicals, which is disfavored in manufacturing and for biomedical applications.

However, all of the afore-mentioned products and approaches have disadvantages. Thus, there is still a need for an alternative that improves orthopedic implant performance at a reasonable cost.

A number of methods have been proposed for the production of scaffolding materials through rapid prototyping-based manufacturing processes, better referred to as layered manufacturing. In this process, a layer is produced, either by curing a liquid polymer (as in stereolithography and solid base curing), inducing a phase change in the material (as in fused deposition modeling, ballistic particle manufacturing, and selective laser sintering of polymers), or removing material (as in conventional machining or laminated object manufacturing). In all of these approaches, a layer is manufactured and then bonded to previously-produced layers, another layer is manufactured and then bonded, etc., until the desired product is produced. When a porous structure is desired, a support structure may be required by the process, which is difficult to remove from the final product. In some processes, unfused powders or poorly fused powders may serve as the support in a porous structure, and these are also difficult to remove from the final product. The final product therefore cannot be produced to the desired porosity, and, for medical device applications, a serious hazard exists that stray particles may contaminate the tissue surrounding the implant, with serious potential health consequences.

All of the polymer-based layered manufacturing approaches have serious shortcomings for soft tissue and bone scaffolding applications. Each suffers from at least one of the following serious deficiencies:
(a) Toxic monomers are included in their chemical formulation;
(b) Sufficiently high porosity cannot be produced;
(c) The desired cell size cannot be produced;
(d) The desired cell morphology cannot be produced;
(e) The required strut thickness cannot be achieved;
(f) The raw materials are very expensive;
(g) The manufacturing process is very slow and complicated; and
(h) The process is restricted to one class of material, usually polymers, and is not well suited for production of another class of materials, notably metals or ceramics.

Rapid prototyping operations in their commercial forms are unable to achieve the required tolerances for scaffold applications. There is some indication that specialized forms of microstereolithography may achieve such tolerances, but stereolithography is notable for its use of toxic monomers that are not fully cured. Furthermore, stereolithography scaffolds are not suitable for in-vivo use.

Production of polymer scaffolding is performed through the layered manufacturing processes described above, or through conventional foam-production techniques. Conventional foam-production techniques include aeration of a molten polymer, inclusion of an aeration element in the polymer which expands the polymer when subjected to heat, and diffusion of gas into the polymer which causes expansion of the polymer when subjected to controlled heating. The main drawback to this type of approach is that the polymers that are preferable for in-vivo use do not lend themselves to such operations.

Direct production of ceramic scaffolding from layered manufacturing approaches involves modifications of the ballistic particle manufacturing or selective laser sintering operations as discussed in Kalpakjian and Schmid, Manufacturing Processes for Engineering Materials, Prentice-Hall, 2002, the entire contents and disclosure of which is hereby incorporated by reference. These processes result in some poorly-fused ceramic particles, or the desired porosity and cell morphologies necessary for scaffolding may not be achieved, especially for larger sections. An indirect method of producing ceramic scaffolding uses a polymer scaffold produced from the methods described above, which is exposed to a ceramic slurry consisting of nano- or micro-scale ceramic particles suspended in water with binders. The ceramic particles coat the polymer precursor, and the structure is then placed in a kiln to fuse the ceramic and remove the polymer. This approach cannot achieve consistent porosity in the interior of thick sections because of the inability of ceramic particles to penetrate into these sections.

Commercialized synthetic bone graft substitutes or scaffolds comprising a porous bioceramic include Pro Osteon™ (Interpore Cross International, Inc., Irvine, Calif.), VITOSS™ (Orthovita, Malvern, Pa.), Norian SRS™ (Synthes-Stratec, affiliates across Europe and Latin America), and Alpha-BSM™ (ETEX Corp., Cambridge, Mass.). Another promising material, ApaPore™ (ApaTech, London, England), is now in clinical trials. Pro Osteon™, VITOSS™ and ApaPore™ comprise monolithic ceramic granules for use as a filler material, whereas Norian SRS™ and Alpha-BSM™ comprise injectable pastes used to fill a space and harden in vivo. All the above materials are calcium phosphates based upon hydroxyapatite (HA), including the more resorbable carbonated apatite and beta-tricalcium phosphate (β-TCP), which is the closest synthetic equivalent to the composition of human bone mineral. Note that ETEX Corp. advertises alpha-BSM as an “amorphous calcium phosphate”; however, the broadened x-ray diffraction peaks are actually indicative of a nanocrystalline apatite phase, not an amorphous material. Over 20 years of research has consistently shown that HA typically exhibits excellent bioactivity and osteoconduction in vivo.

The longstanding industry benchmark for these materials is Pro Osteon™ (FIG. 1), which utilizes coralline calcium carbonate fully or partially converted to HA by a hydrothermal reaction, see D. M. Roy and S. K. Linnehan, Hydroxyapatite formed from Coral Skeletal Carbonate by Hydrothermal Exchange, Nature, 247, 220-222 (1974); R. Holmes, V. Mooney, R. Bucholz and A. Tencer, A Coralline Hydroxyapatite Bone Graft Substitute, Clin. Orthop. Rel. Res., 188, 252-262 (1984); and W. R. Walsh, P. J. Chapman-Sheath, S. Cain, J. Debes, W. J. M. Bruce, M. J. Svehla and R. M. Gillies, A resorbable porous ceramic composite bone graft substitute in a rabbit metaphyseal defect model, J. Orthop. Res., 21, 4, 655-661 (2003), the entire contents and disclosures of which are hereby incorporated by reference. However, variations in the coralline feedstock give rise to architectural and compositional variations which may be problematic for reliable mechanical integrity and biocompatibility. VITOSS™, Norian SRS™ and Alpha-BSM™ possess mechanical strength far inadequate for most orthopaedic applications. These materials, as well as calcium sulfate materials, are designed on the premise of rapid scaffold resorption. Furthermore, all the above materials suffer from low fracture resistance (brittleness), leading to the risk of catastrophic failure prior to healing. The key to mitigating the inherent brittleness of a ceramic biomaterial lies in the proper design of the scaffold architecture. With the possible exception of ApaPore™, which uses a porogen to form a controlled porosity network, the scaffold architecture of the above materials cannot be specifically tailored.

Developmental studies have recently begun to investigate the fabrication of tailored HA scaffolds using various direct-write processes including solid free-form fabrication, extrusion-based robotic deposition (also referred to as “robocasting”), and three-dimensional printing (3DP), see T. M. G. Chu, J. W. Halloran, S. J. Hollister and S. E. Feinberg, Hydroxyapatite implants with designed internal architecture, J. Mater. Sci: Mater. Med., 12, 471-478 (2001); T. M. G. Chu, D. G. Orton, S. J. Hollister, S. E. Feinberg and J. W. Halloran, Mechanical and in vivo performance of hydroxyapatite implants with controlled architectures,” Biomaterials, 23, 5, 1283-1293 (2002); and J. E. Smay, G. M. Watson, R. F. Shepherd, J. Cesarano III and J. L. Lewis, Directed Colloidal Assembly of 3D Periodic Structures, Adv. Mater., 14, 18, 1279 (2002); the entire contents and disclosures of which are hereby incorporated by reference. These processes are all derived from rapid prototyping which, as the name implies, are excellent for prototypes, but often lack the production rates necessary for manufacturing feasibility. Furthermore, these processes are all limited to geometric architectures (cylindrical rods, plates, etc.) and are not easily adapted to resemble the trabecular architecture of bone, which has been shown to enhance osteoconduction in metallic implants (Hedrocel®, Implex Corporation).

Since bioceramic scaffolds are inherently limited by a relatively low fracture toughness, rapid osteoconduction and osteointegration are crucial to clinical success. Many investigations have documented and continue to study the influence of the porosity fraction, size and morphology on osteoconduction. As noted above, consensus is generally aimed at mimicking the architecture of trabecular bone (FIG. 3). The composition of the bioceramic has also been known to play a significant role. Recent studies have elucidated the detrimental and beneficial effects of very minor amounts of impurities and dopants. Parts per million levels of lead, arsenic, and the like, are commonly present in commercial water supplies and, if incorporated into hydroxyapatite, may lead to inhibition of osteoconduction. On the other hand, carbonated apatite exhibits faster bioresorption than pure HA, if desired, and 1-3 wt % silicon additions to HA have shown a two-fold increase in the rate of osteoconduction over pure HA, see N. Patel, I. R. Gibson, K. A. Hing, S. M. Best, P. A. Revell and W. Bonfield, A comparative study on the in vivo behaviour of hydroxyapatite and silicon substituted hydroxyapatite granules, J. Mater. Sci: Mater. Med., 13, 1199-206 (2002); and A. E. Portera, N. Patela, J. N. Skepperb, S. M. Besta and W. Bonfield, Comparison of in vivo dissolution processes in hydroxyapatite and silicon-substituted hydroxyapatite bioceramics, Biomaterials, 24, 4609-4620 (2002), the entire contents and disclosures of which are hereby incorporated by reference. Silicon-doped HA is being developed at ApaTech, under the name Pore-SI.

Finally, the above discussion has been limited to bulk (monolithic or injectable), porous HA. Plasma sprayed HA coatings on smooth, roughened, or porous metallic implants have received huge investments in time and resources, yet the mechanical integrity and adhesion of the coating to the metal remain as stumbling blocks.

Production of open-celled metal scaffolding suitable for tissue in-growth is limited to CVD onto pyrolized polymer precursors (as with HEDROCEL), production of metal foams by forcing hot air into molten metal and solidifying the resultant froth, through powder metallurgy techniques (sometimes combined with chemical agents that expand the microstructure and increase porosity during sintering), or by leaching a two-phased metal. The CVD process produces a high-quality scaffold, but the process is expensive, environmentally hazardous, time consuming and has high scrap rates. None of the other processes have been successful in producing optimal porosity or cell sizes for scaffold applications.

### SUMMARY

It is therefore an object of the present invention to provide a bone in-growth material that improves orthopedic implant performance at a reasonable cost.

According to a first broad aspect of the present invention, there is provided a bone and tissue in-growth and on-growth scaffolding, comprising bonded layers of material, wherein the material comprises at least one of a metal, a ceramic and a polymer, wherein the material has a porosity between about 5% and about 95%, has cells of mean spacing between about 0.05 mm and about 5 mm and has about 0.05 mm to about 2 mm thick struts.

According to second broad aspect of the invention, there is provided a method for producing a bone and tissue in-growth scaffolding, comprising providing sheets of machined material, wherein the material comprises at least one of a metal, a ceramic and a polymer, wherein the material has a porosity between about 5% and about 95%, has cells with mean spacing between about 0.05 mm and about 5 mm, and has about 0.05 mm to about 2 mm thick struts; subjecting the sheets to compression; and bonding the sheets to produce a bone and tissue in-growth scaffolding.

Other objects and features of the present invention will be apparent from the following detailed description of the preferred embodiment.

### BRIEF DESCRIPTION OF THE DRAWINGS

The invention will be described in conjunction with the accompanying drawings, in which:

FIG. 1 shows a three dimensional image from micro-computed tomography (μCT) showing a section of a Pro Osteon™ granule. Note that the section is approximately 1 mm in height;

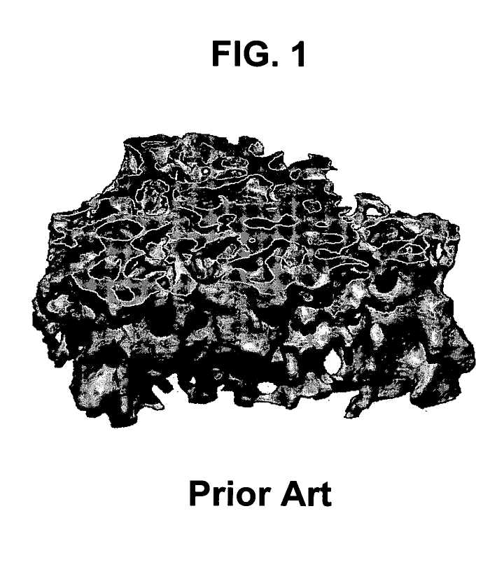

FIG. 2. is a schematic diagram of pressurized tape infiltration, showing the infiltration of a porous “negative” polymer tape with ceramic slurry;

FIG. 3 is a CT scan of bone;

FIG. 4 shows a slice of bone obtained from a CT scan file;

FIG. 5 shows a modified material geometry in accordance with an embodiment of the present invention to facilitate use of described manufacturing process; and

FIG. 6 shows reassembled slices of material to form a structure in accordance with an embodiment of the present invention based on tissue structure.

### DETAILED DESCRIPTION

It is advantageous to define several terms before describing the invention. It should be appreciated that the following definitions are used throughout this application.

Definitions

Where the definition of terms departs from the commonly used meaning of the term, applicant intends to utilize the definitions provided below, unless specifically indicated.

For the purposes of the present invention, the term “bone in-growth” refers to a material's ability to allow or encourage the formation of bone tissue into and onto a porous scaffold to achieve a strong intimate junction and superior fixation.

For the purposes of the present invention, the term “bone on-growth” refers to apposition of bone tissue on the surface of a material. It is differentiated from bone in-growth in that the bone does not typically infiltrate past the immediate surface layer.

For the purposes of the present invention, the term “porosity” refers to a property of a material as defined by the apparent volume minus the actual volume, then divided by the apparent volume.

For the purposes of the present invention, the term “cell shape” refers to the morphology, shape and size of the pores in a material.

For the purposes of the present invention, the term “strut” refers to the structural members, either rods, beams, plates, shells or columns, that define the face or edge of a cell within a cellular solid material.

For the purposes of the present invention, the term “implant” refers to any device that is placed inside the human body.

For the purposes of the present invention, the term “diffusion bonding” refers to joining of materials through application of heat and pressure without causing a phase change in either of the materials, and without the use of a filler material.

For the purposes of the present invention, the term “simultaneously” means at the same time or in the same step of a process.

For the purposes of the present invention, the term “barrier layer” refers to a solid section next to one or more porous sections of a material, which prevents exposure of material on one side of the solid section to the materials or environment on the other side of the solid section.

For the purposes of the present invention, the term “slag” refers to vitreous materials generally containing impurities, and/or oxide and resolidifed molten metal droplets.

For the purposes of the present invention, the term “open-celled structure” refers to a porous structure with very large permeability, and where no significant surface barriers exist between cells. In particular, an example of a fully open-celled structure is trabecular metal, and an example of a partially open-celled structure is a typical polymer foam.

For the purposes of the present invention, the term “fully closed-cell structure” refers to a porous material where the pores are not connected; thus the material has zero permeability.

For the purposes of the present invention, the term “transition” refers to a change from one state or condition to another, typically in a gradual fashion, such as a transition from an open-celled structure to a fully closed-cell structure in a material of the present invention.

Description

Laser machining, chemical machining or etching, photochemical machining, plasma etching, stamping, electron beam machining and textile manufacturing processes are capable of producing extremely porous and controlled thin parts. As an example, the manufacture of laser machined stents is described in Kalpakjian et al., see Kalpakjian, S., et al., 2003, Manufacturing Processes for Engineering Materials, New York, Prentice-Hall, the entire contents and disclosure of which is hereby incorporated by reference. In Kalpakjian et al., the struts of the stent are as narrow as 91 μm, which is similar to the struts in Implex's trabecular metal, which are roughly 100 μm to 300 μm.

Laser machining, chemical machining or etching, photochemical machining, plasma etching, electron beam machining, stamping, and textile manufacturing processes are difficult to apply to rapid prototyping operations, especially when metals or ceramics are involved. It is difficult to produce a single layer with any of these processes, but once setup in accordance with the teachings of the present invention, they may all produce many layers simultaneously or separately from the bonding operation. Therefore, according to an embodiment of the present invention, all layers of a material are produced, treated as necessary, and then all layers are joined simultaneously, instead of sequentially, as in rapid prototyping operations. This is a fundamental difference between this embodiment of the present invention and commercial rapid prototyping operations.

Thus, a manufacturing approach according to an embodiment of the present invention for producing bone in-growth material involves the following steps:
(a) Sheets of at least one metal, polymer, ceramic or composite are machined, for example, through laser machining;
(b) Any slag, splatter, maskant or other contaminants that result from laser machining may be removed, for example, through chemical machining or an equivalent process;
(c) The sheets are stacked in a mold produced from graphite or other suitable material to produce the desired product. The graphite or other suitable material preferably has a higher melting temperature than the sheet material and, according to embodiments of the present invention, is preferably chemically inert with respect to the sheet material at elevated temperatures;
(d) The mold is tightened to subject the layers to compressive stress sufficient to compress the layers, but not large enough to cause significant plastic deformation;
(e) The mold is placed in a vacuum furnace, or other heating means, to diffusion bond the sheet layers. The temperatures used in diffusion bonding vary by material, but are roughly 90% of the melting temperature on an absolute temperature scale.

After diffusion bonding, the resultant material is as porous as the layers from which it was constructed. The porosity is controllable from zero porosity to essentially a fully porous material. While bone in-growth materials such as sintered metal wires and beads produce porosities of roughly 20%, the materials according to the processes of the present invention may easily achieve a porosity of from about 5% to about 95%, and may achieve infinitesimally small porosities or porosities approaching 100%. The pore size may be as small as achievable by the machining processes (roughly 10 nanometers for thin foils) and may be very large, for example, hundreds of millimeters in diameter. This process allows for a versatility and control in the material microstructure and porosity that is unmatched by any other known manufacturing process.

In an embodiment of the present invention directed to tissue scaffolding, the expected porosity is between 50 and 90%, preferably between 70 and 90%, and in some embodiments between 70 and 80% with a mean cell spacing of about 0.05 mm to about 5 mm, preferably between 0.25 mm and 1.0 mm, and in some embodiments between about 0.3 mm and about 0.6 mm. A scaffolding according to the present invention may also have struts that are between about 0.05 mm and about 2 mm thick, preferably between about 0.08 mm and 0.3 mm thick.

Alternatively, material sheets according to an embodiment of the present invention may be produced by chemical etching, photochemical blanking, electroforming, stamping, plasma etching, ultrasonic machining, water jet cutting, electrical discharge machining or electron beam machining of individual layers, or a porogen that is removed by dissolution (e.g., salt), melting (e.g., lost wax), or pyrolysis. Details of these processes are discussed further below.

In chemical etching, a sheet of the desired material has a desired pattern printed onto it, known as the resist. The resist-covered material is then placed in an aqueous bath containing chemicals needed for dissolving the target material, but in which the resist is insoluble. Wherever the sheet is coated by the resist, the material is protected, but where it is exposed, the material is dissolved by the chemical bath.

Photochemical etching is similar to chemical etching, except that the resist pattern is achieved by curing or baking the resist preferentially, using light energy.

Stamping involves pressworking operations such as shearing and stretch forming that may produce the desired pattern through direct action of a die set.

Electrical discharge machining uses the heating action of an arc in a dielectric fluid between an electrode and the electrically conductive workpiece. The arc melts a small volume of the workpiece. The arc then collapses and the associated microscopic cavitation results in particles to be suspended in the dielectric fluid. The clearance between the electrode and workpiece is carefully controlled, and the sheet profile is produced that matches the electrode shape.

In ultrasonic machining, abrasive particles impact the workpiece as a result of the agitation from a vibrating tool. A resist pattern placed on the workpiece restricts the resulting machining to unprotected regions as in chemical etching described above.

In plasma etching, the workpiece is placed in an evacuated chamber where a plasma, commonly fluorine gas, is charged and machines the workpiece. As with chemical etching, a resist defines the resulting workpiece shape.

Electroforming involves the production of a resist, followed by electroplating or electroless plating or a combination of these approaches to produce the desired layer.

Water jet cutting uses the abrasive action of a high velocity water jet to remove workpiece material. The water jet is highly focused, and controlled by a gantry robot or equivalent, allowing control of the machined geometry.

Electron beam machining uses focused beams of electrons to remove material from an electrically conductive material. It is similar to laser machining, except that the energetic beams consist of electrons instead of light.

The layers may be produced by using a laser, rotary die or mechanical press to machine slits in the layers and then subjecting the layers to an expansion process before diffusion bonding. The expansion process involves placing the sheet in a state of tension sufficient to cause plastic deformation in the sheet. Because of the pre-machined slits or other features, the resultant sheet develops a porosity and a controlled morphology. This has the benefit of reducing the amount of wasted material. For example, for scaffolds with desired porosities of 80%, 80% of the material needs to be removed as material scrap, which adds to the product cost. By machining slits and then expanding the material, the desired porosities may be achieved without high material scrap rates.

The layers may be produced by knitting or weaving threads of the material, using processes common in the textile industry.

Adhesive bonding or other suitable bonding means such as friction welding, ultrasonic welding, cold welding, laser welding, resistance welding, arc welding, brazing, glazing, etc. may be used to join the layers or to attach the material to a solid surface.

In embodiments, the present invention utilizes thin foils, approximately 10 μm thick to sheets 2 mm in thickness, to produce highly porous material. The material may be produced in bulk form using many layers of foil or sheets. Such an approach is suitable for any thickness of material.

Another embodiment of the present invention produces pads of material approximately 2-3 millimeters thick, which may then be plastically deformed and bonded or joined to implants. The present invention may, in embodiments, produce pads from about 0.5 mm to about 5 mm thick. The thickness is not restricted by the process, but bone in-growth of a few millimeters is sufficient for good fixation.

Material according to embodiments of the present invention has numerous advantages discussed further below.

Material of the present invention may be any metal that may be rolled into foil (titanium, cobalt-chrome, tantalum, stainless steel, magnesium, or any other ductile metal), plated into a foil shape through electroforming or produced into foil by any other means. Thus, any metal or metal alloy may be processed by methods of the present invention.

Material of the present invention may be any polymer or reinforced polymer, such as nylon, polycarbonate, polymethylmethacrylate, polyethylene, polyurethane, polyaryl etherketone, polyetheretherketone, polylactide, polyglycolide polylactide-co-glycolide and synthetic or natural collagen etc., which may be shaped into a film by blow molding, dip coating, solvent casting, spin coating, extrusion, calendaring, injection molding, compression molding or any other suitable process. Examples of bioresorbable thermoplastics applicable to the manufacturing process described herein include, but are not limited to, poly(DL-lactide) (DLPLA), poly(L-lactide) (LPLA), poly(glycolide) (PGA), poly(ε-caprolactone) (PCL), poly(dioxanone) (PDO), poly(glyconate), poly(hydroxybutyrate) (PHB), poly(hydroxyvalerate (PHV), poly(orthoesters), poly(carboxylates), poly(propylene fumarate), poly(phosphates), poly(carbonates), poly(anhydrides), poly(iminocarbonates), poly(phosphazenes), and the like, as well as copolymers or blends thereof, and combinations thereof.

Examples of non-bioresorbable thermoplastics applicable to the manufacturing process described herein include, but are not limited to, polyethylenes, such as high density polyethylene (HDPE), ultra high molecular weight polyethylene (UHMWPE), and low density polyethylene (LDPE), as well as polybutylene, polystyrene, polyurethane, polypropylene, polyaryletherketone, polyacrylates, polymethacrylates, such as polymethylmethacrylate (PMMA), and polymerized monomers such as tri(ethylene glycol) dimethacrylate (TEG-DMA), bisphenol a hydroxypropyl methacrylate (bis-GMA), and other monomers listed herein below, and the like, as well as copolymers or blends thereof and combinations thereof.

According to an embodiment of the present invention, an exothermic phase-change polymer may be incorporated into an implant adjacent to the scaffold. Such a polymer may create excess thermal energy, and thus, in an embodiment of the present invention, an evacuated or partially evacuated layer may be provided on the scaffold to provide a protection against thermal damage to bone or other tissue. An evacuated or partially evacuated layer has space in the layer for thermal insulation.

Material of the present invention may be any ceramic, such as alumina, partially stabilized zirconia, hydroxyapatite (HA)—including HA doped with one or more of the following: Si, Mg, carbonate, and the like, calcium phosphates and the like, etc., that may be shaped into a film by tape casting, doctor blade process, robocasting, jiggering or any other process. Thus, any ceramic may be processed by methods of the present invention.

A ceramic layer may be microtextured by laser ablation, chemical etching, photochemical etching, or ultrasonic machining. The layers may be stacked as desired. This is followed by a firing step, where the adjacent layers are fused to form a material.

According to an embodiment of the present invention, ceramic particles, whiskers or fibers may be deposited on a scaffold. If the scaffold is, for example, metal, the ceramic deposited on the metal scaffold forms a hybrid scaffold.

Material of the present invention may be a composite material of metals, plastics and/or ceramics and may be processed by methods of the present invention. A continuous or discontinuous fiber reinforced composite material produced by a method of the present invention may have any volume fraction of reinforcement desired.

A hybrid metal-ceramic material may be produced by manufacturing a metallic scaffold. This scaffold may then be placed in a reaction chamber for producing hydroxyapatite or other ceramic material, and the ceramic may bridge struts of metal that are in close proximity to one another. When removed from the reaction chamber, the material consists of a continuous metallic scaffold and discontinuous ceramic struts between struts of metal.

Material of the present invention may be of natural origin, e.g., animal tissue or vegetable products.

Material of the present invention may be bioactive or passive. Such a material may contain growth factors, antibiotics, steroids and the like. A material of the present invention may be a bioresorbable polymer or a combination of materials.

Ceramic material according to an embodiment of the present invention may also be produced using a polymer precursor. In an embodiment of the present invention, ceramics or metal powder materials according to the present invention may be produced using a polymer precursor and subsequent slurry infiltration of the precursor. Conventional slurry infiltration may be done, but it is generally done one layer at a time. Methods of the present invention allow multi-layer infiltration and more uniform distribution of ceramic in a layer and throughout the thickness of the material. This eliminates the problem of suspect porosity or poorly fused material below the surface of porous materials.

A porous ceramic layer may be produced using a polymer or metal precursor. The precursor may be a “negative image” of the desired material that may be infiltrated with ceramic slurry in a doctor blade process, or, in an alternative embodiment, it may be a “positive image” of the desired morphology and infiltrated by dipping it into an inviscid slurry of water and suspended ceramic or metallic powders. The layers may then be stacked, compressed and fired to fuse particles and layers, resulting in a material with controlled microstructure (morphology and porosity). These approaches ensure that a ceramic material may be produced with uniform microstructure and porosity throughout a bulk shape, if desired. Further, these approaches allow designed variations in the microstructure and porosity at any location within a volume.

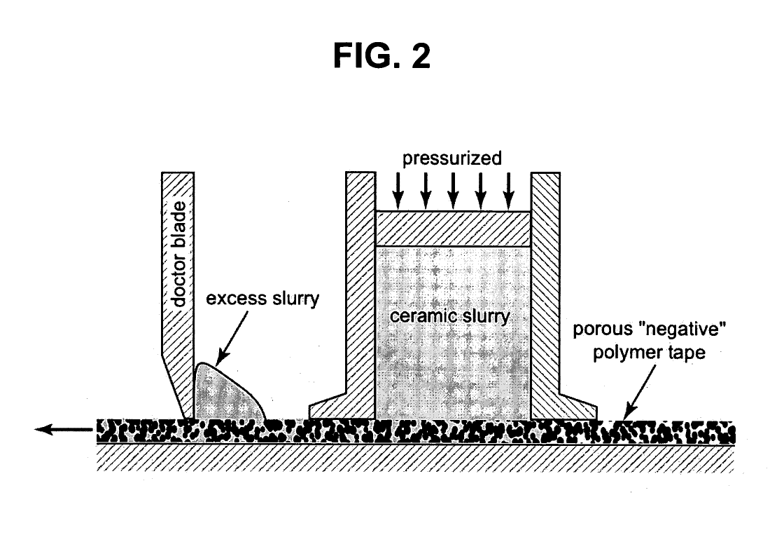

One method of use for the present invention is termed pressurized tape infiltration, which comprises an adaptation of conventional tape casting where a porous “negative” polymer tape is infiltrated with a ceramic slurry (FIG. 2). The infiltrated tapes may then be cut, stacked and/or pressed and shaped prior to sintering the ceramic. Upon sintering the ceramic, the ceramic phase is densified, the layers are diffusion bonded, and the polymer tape is pyrolized, leaving a pore network defined by the original polymer tape. Sintering may be pressureless or pressure assisted.

The advantages of pressurized tape infiltration and material made therefrom over conventional methods and materials include:
1) A semi-continuous process that is suitable for large-scale manufacturing in contrast to the batch processes used for all other materials and methods. This includes Pro Osteon™, VITOSS™, Norian SRS™, Alpha-BSM™, ApaPore™, and Hedrocel®, as well as direct-write (rapid prototyping) processes under development;
2) Improved infiltration of a thin porous tape versus bulk polymer scaffolds. Previous methods used to infiltrate a porous polymer scaffold with a ceramic slurry suffered from the inherent difficulty of complete and uniform infiltration of a bulk scaffold with a relatively high viscosity ceramic slurry;
3) Laminated object manufacturing may be used to tailor the macroscopic shape and microscopic architecture of a material by sequentially stacking infiltrated tapes in their flexible, presintered (“green”) state. For example, layers may incorporate a changing pore architecture (functional gradient). Green layers may also be stacked and pressed to conform to a surface contour, such as that used for fixation by bone in-growth on an implant surface; and
4) The ability to produce and tailor trabecular architectures based upon the polymer “negative”. This has not been accomplished in any ceramic scaffold to date.

Methods of the present invention may be used to produce layers of scaffold material that are subsequently coated with another material by chemical vapor deposition, physical vapor deposition, sputtering, plasma or metal spray, using sol-gel techniques, electroplating, mechanical plating or any other plating technique. Therefore, the material may have a coating of diamond, diamond-like carbon, aluminum oxide, other ceramics or cermets, a metal or metal alloy, a polymer, or a nanometer-scale thick coating of biologic material, including animal, vegetable or human tissue.

Material of the present invention may be a shape memory alloy, such as a nickel alloy. An advantage of using a shape memory alloy is that the shape memory alloy may be deformed into a deployable shape, placed inside a prepared cavity within the body and then allowed to return to the initial, desired shape for the implant.

Material of the present invention may be produced by wrapping the sheets or layers around a graphite mandrel and then diffusion bonding the material. This method according to an embodiment of the present invention provides for the production of hollow shapes suitable for applications, such as spinal cages and the like.

According to an embodiment of the present invention, the shape of the pores may be controlled by the patterns machined by a laser or other layer manufacturing method. There is no realistic restriction on the pore shapes that may be constructed.

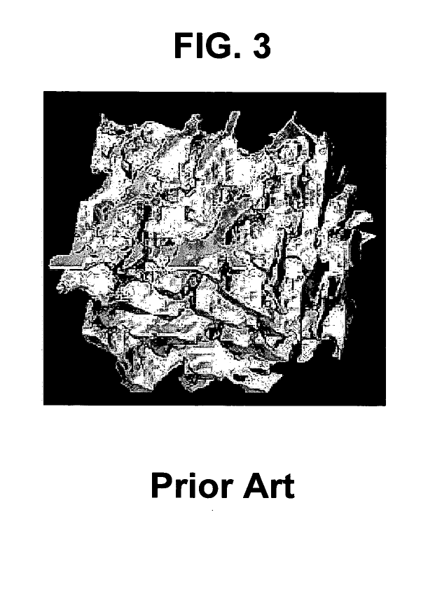

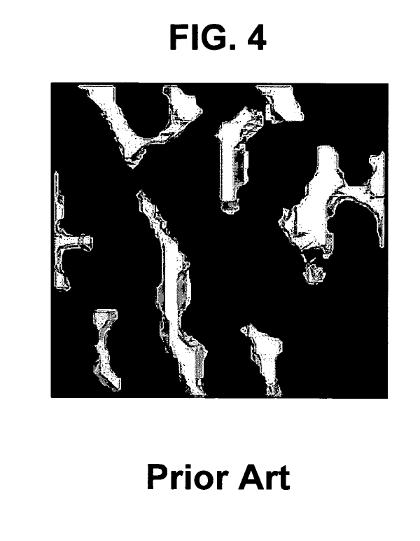

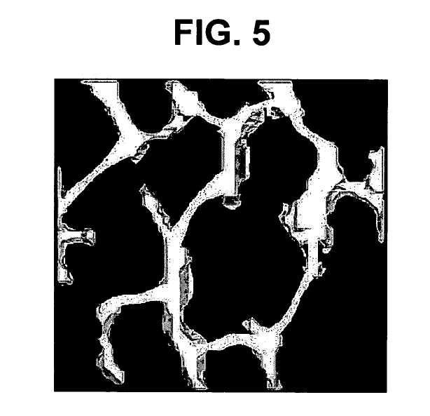

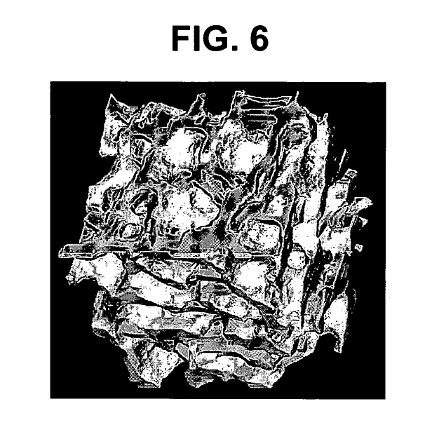

According to an embodiment of the present invention, a biomimetic scaffold may be produced, wherein the material morphology closely matches that of tissue. For example, a micro-computed topography (micro-CT) scan of trabecular bone may be reproduced in the material. The geometry may be modified to add struts and/or remove features. For example, FIG. 3 shows the results of a CTscan of bone, while FIG. 4 shows a slice obtained from the CTscan. FIG. 5 shows a modified geometry in accordance with an embodiment of the present invention, in which struts have been added and selected overhangs have been trimmed to provide an attractive surface for bone in-growth. The struts may then be blended from layer-to-layer to obtain a smoothly transitioned three-dimensional object when all slices are joined. FIG. 6 shows the slices when reassembled in accordance with an embodiment of the present invention. Within the computer software, the material is reflected along three Cartesian planes and joined to the original shape to form a “brick” of material that may be used to assemble a volume of scaffold. This process may be used to produce scaffolds that mimic the geometry of any tissue.

According to an embodiment of the present invention, a biomimetic scaffold may be produced, wherein the material morphology and/or mechanical properties closely match that of tissue by manipulating the design as a CAD file. The struts may be enlarged or reduced in cross-section, and the complete volume analyzed to predict the mechanical properties such as stiffness, strength, permeability, porosity, etc. The geometry may be modified in order to duplicate the mechanical properties of the tissue it is intended to contact.

Material of the present invention may be produced in any desired shape.

The porosity of the material of the present invention may be tightly controlled to obtain a desired value; the bulk density of the material may range from very small to fully dense. Layers may have a desired shape produced in them, and then stacked in no particular order, where the layers are not directly over one another but instead are offset a random distance to obtain a random stacking.

Layers may have a desired shape produced in them and then stacked so that there is a designed transition from one layer to the next, allowing a three-dimensional geometry. This may be achieved by using geometrical features in the sheets that facilitate stacking, such as pin holes, flats, or other stackable, indexable features.

The porosity of the material of the present invention may be graded through the thickness of the material. This may be accomplished by producing layers with different porosities and stacking them in a desired fashion to provide the desired transition from layer to layer. For example, a fully dense material may have directly above it a material with, for example, 10% porosity, followed by 20% porosity, etc., until the top layer or a pad is substantially porous, or, in an alternative embodiment, is fully dense to facilitate bonding to a metal orthopedic implant core. This embodiment of the present invention has the advantage of providing a bone in-growth, porous material for bone-contact, while producing a solid or near-solid material for superior bonding to an implant core structure.

The material properties, porosity and structure of material of the present invention may be graded through the thickness to mimic the transition between naturally occurring structures within the body. For example, in an embodiment of the present invention, one end of an implant may have a structure designed for integration with bone and the other end for soft tissue.

According to an embodiment of the present invention, a solid layer may be used to maintain fluid under pressure within a scaffold. For example, a solid layer may define a pressure vessel to encompass a pressurized fluid. Such a solid layer may comprise metal, composite, a flexible polymer, etc. A solid layer may also be compressible or foldable, and then expandable by internal pressurization. A compressible solid layer may, in an embodiment of the present, also be configured to contain a pressurized fluid.

The material of the present invention provides a natural vehicle for introduction of biological materials and growth factors. This embodiment of the present invention presents a superior topography and density for the integration of bioactive materials. Such an embodiment may take the form of a biologic material that is incorporated directly, such as a bioresorbable polymer that contains or encapsulates growth factors or other medications. Such an embodiment may also take the form of a biomaterial or growth factor, antibiotic, steroid and the like that is encapsulated within the material. In this form, a barrier layer may be designed of a resorbable material, or a partial barrier with controlled permeability may be produced to control the release of the biomaterial, growth protein, antibiotic, steroid and the like.

The present invention also provides for bone in-growth implant designs that may be obtained by producing a material with three regions: an outer region with bone in-growth porosity and cell shape, a central region with a stiffness that closely matches trabecular bone (roughly 3 GPa elastic modulus), and a solid metal core. This allows the stiffness of the implant to be tailored so that stress shielding of bone does not occur, bone in-growth is optimized, and, as a result, a vigorous and healthy bone may be maintained.

Further embodiments of the present invention provide for layered scaffolds serving various purposes. For example, a material of the present invention may have a layer with a finite thickness intended to integrate with tissue, beneath which is a layer designed to contain and control the release of a medicine encapsulated by a bioresorbable material. Beneath this bioresorbable material layer may be a transition to a solid layer suitable for bonding to a solid core, or that comprises the implant core.

In an alternative embodiment, a material of the present invention may have a layer with a finite thickness intended to integrate with tissue, beneath which may be a layer that transitions to a fully dense layer or layers. Beneath this may a finite layer that has a material and microstructure intended to bond with an injection molded polymer. This arrangement is intended for insert injection molded implants where the liquid polymer cannot permeate through the tissue in-growth thickness.

In an alternative embodiment, a material of the present invention may have a layer with a finite thickness intended to integrate with tissue, beneath which may be a layer that transitions to a fully dense layer or layers. Beneath this is a finite layer that has a material and microstructure intended to bond with other tissue. This arrangement is intended for implants where controlled depth of in-growth is desired on each side of the solid layers.

The present invention is well-suited for surgeries such as facial reconstruction, since the material thickness may be contoured to match the particular patient's anatomical features, and the microstructure of the material may be simultaneously optimized to encourage tissue in-growth and healing.

The present invention also provides for the production of a scaffold comprising two or more materials. For example, a 2 mm pad may be produced by creating 1 mm of the scaffold from titanium and 1 mm of the scaffold from a bio-compatible polymer. A boundary film of the polymer may be partially melted into the metal scaffold, and the polymer scaffold portion may then be attached to the exposed polymer layer or the metal scaffold portion.

Material of the present invention may use a barrier layer. The material may consist of a surface intended for integration with tissue, a transition to a solid layer, and a transition to a geometry designed for integration with material on the other side of the material from the tissue. For example, a barrier layer of the present invention may contain or surround a liquid polymer introduced by insert injection molding or one that cures within the body, such as with the Zimmer T2™ hip fracture and bone plate implants.

When there is a transition between tissues, such as soft tissue/bone attachment, a barrier layer of the present invention may define the in-growth limits of the two tissues. Thus, if the soft tissue grows faster than the bone, space is available for the bone to continue growing into the scaffold and there is enough room for the soft tissue to become established.

A barrier layer of the present invention may provide a thermal insulating layer, for situations in which an exothermic polymer, such as polymethyl methacrylate and the like, is placed on one side of the material. In particular, the barrier layer may be an insulating material; it may be extremely porous or evacuated. Thus, the barrier layer may provide protection against thermal necrosis from a curing polymer.

The barrier layer may have defined permeability to allow controlled release of bone morphogenetic proteins (BMPs) or growth factors, such as the TGF-beta superfamily (e.g., TGF-β, bone morphogenic proteins, such as BMP-2, BMP-7, and the like), fibroblast growth factors, vascular endothelial growth factors, insulin-like growth factors, interleukins, transcription factors, matrix metalloproteinases to enhance tissue asperity regeneration, or proteins such as oseopontin, integrins, matrix receptors, RGB and the like, and drugs such as bisphosphonates (e.g., alendronate, risendronate, etc.), hormones such as estrogen, parathyroid hormone (PTH), vitamins/minerals such as calcium, selective estrogen receptor modulators such as raloxifene, human growth hormone, 1,25-(OH)D₃ (vitamin D₃ and vitamin D). These medications may then be supplied in essentially bulk form behind the barrier layer and may utilize controlled delivery based on the permeability of the barrier layer.

A barrier layer of the present invention may define a pressurized volume, for embodiments such as biomimetic spine disk replacements using a gel as the artificial nucleus. Natural spine disks use a pressurized viscous fluid in which the nucleus is contained by an annulus. As the spine is loaded in compression, the nucleus damps vibrations and is pressurized by the annulus. The barrier layer allows the use of liquids in a nucleus, and preserves the biological function of the nucleus and annulus.

According to an embodiment of the present invention, a scaffold may be constructed in whole or in part of a piezoelectric material. Suitable piezoelectric materials include quartz, barium titanate, rochelle salt, lead zirconium titanate (PZT), lead niobium oxide, polyvinyl fluoride, etc. A piezoelectric material generates a voltage when subjected to mechanical stress, and generates a mechanical stress when subjected to a voltage.

In accordance with an embodiment of the present invention, a scaffold may comprise a piezoelectric material encapsulated by another material. For example, such a piezoelectric material may be encased by a metal, polymer or ceramic, and thereby incorporated into a scaffold of the present invention without having direct tissue contact.

A piezoelectric material may be textured as described herein, or it may be a separate structure surrounded by textured material.

A piezoelectric material as described above may be placed toward the bone surface of a scaffold. When a voltage is applied to the piezoelectric material, the piezoelectric material stresses the scaffold and therefore the bone. Such mechanical stresses are known to be important for bone in-growth.

In accordance with an embodiment of the present invention, a piezoelectric material may be attached to the implant, so that a voltage cycle is encountered with every loading. For example, if attached to a hip stem or knee implant during walking, a voltage pulse may be applied that corresponds to the time during foot contact with the ground.

Voltage may be stored or applied to a different piezoelectric material elsewhere in the scaffold to cause a stress where desired. Alternatively, a control circuit may be incorporated into the scaffold structure that applies a desired stress cycle. The stress cycle applied may mimic the biological loading of bone, regardless of the stiffness of the implant. Thus, stress shielding, a problem commonly encountered with large metal implants, may be eliminated in this fashion.

In accordance with an embodiment of the present invention, a control circuit for a piezoelectric material may incorporate a transformer coil, so that an external power source in the form of a magnetic field may be used to actuate the scaffold. Such an arrangement would allow a patient to apply a power source in the form of a pad or equivalent structure to the outside of the body. The power transferred to the scaffold then stresses the bone and encourages bone in-growth.

Deformation of a piezoelectric material in accordance with an embodiment of the present invention may be used to generate electrical stimulation of bone or tissue to encourage healing and in-growth at the surface.

The present invention is applicable to orthopedic implants, dental implants, bone in-growth surfaces, soft tissue scaffolding, etc.

In an embodiment, the material of the present invention is suitable for cemented implants. The implant may comprise a metal core, with a layer adjacent to the core that may be fully or partially constructed from polymethyl methacrylate, or may encapsulate a polymethyl methacrylate monomer with a metal layer as described above. For cemented implant designs according to embodiments of the present invention, a bone cement mantle may permeate into the porous material at the exterior, and contact the barrier layer. The porous material at the exterior may be a high molecular weight polymer that dissolves in the bone cement because of the materials' large surface area to volume ratio. The bone cement provides a catalyst, usually in the form of cleaved benzoyl peroxide, that dissolves the barrier layer, exposing the monomer and causing it to cure. The resultant curing yields an implant with a metal-, polymer- or ceramic- reinforced bone cement layer, which increases the bond strength and durability of the cemented implant. The embodiment when fully cured comprises a metal or other material as the core of the implant, followed by a layer of metal reinforced polymer, followed by a layer of high molecular weight polymer, followed by conventional bone cement, followed by bone. The porosity and cell morphology are different for bone cement penetration than that for bone in-growth, but the manufacturing method described by the present invention is capable of producing the morphology and porosity desired. Since bone cements have limited adhesive strengths against metallic implants, this reinforced layer and graded stiffness and strength leads to superior bonding.

All other manufacturing methods for bone or tissue scaffolds are restricted to one or several materials. Usually, the processes are restricted to polymers. The embodiments of the present invention allow the development of the same morphology and density regardless of material. This allows construction of a consistent scaffold design from a variety of materials to suit the surgeon's preference and the patient's needs. Methods of the present invention allow production of the same shape of scaffold from polymers, metals, ceramics, biologic materials or composites, or any combination of these materials.

Polymers generally do not have the required mechanical properties to serve as tissue scaffolding unless reinforced by other materials. None of the existing prior techniques produce polymers with the desired volume fraction of reinforcement. The manufacturing methods of the present invention produce porous scaffolds of reinforced polymers in which the reinforcement has the desired volume fraction of reinforcement. For example, a polyetheretherketone sheet reinforced by, for example, continuous high tenacity graphite fibers or discontinuous hydroxyapatite crystals may be laser machined to the desired layer geometry, stacked and joined as described above. The sheet is not limited to any particular matrix or fiber material, nor is it limited to fiber volume reinforcement percentages.

All documents, patents, journal articles and other materials cited in the present application are hereby incorporated by reference.

Although the present invention has been fully described in conjunction with the preferred embodiment thereof with reference to the accompanying drawings, it is to be understood that various changes and modifications may be apparent to those skilled in the art. Such changes and modifications are to be understood as included within the scope of the present invention as defined by the appended claims, unless they depart therefrom.

1. A bone and tissue in-growth and on-growth scaffolding, comprising bonded layers of material, wherein said material comprises at least one of a metal, a ceramic and a polymer, wherein said material has a porosity between about 5% and about 95%, has cells of mean spacing between about 0.05 mm and about 5 mm and has about 0.05 mm to about 2 mm thick struts.

2. The scaffolding of claim 1, wherein said cells are equiaxed.

3. The scaffolding of claim 1, wherein said cells are elongated.

4. The scaffolding of claim 1, wherein said porosity is between about 70% and about 90%.

5. The scaffolding of claim 1, wherein said cells have mean spacing of between about 0.25 mm and 0.6 mm.

6. The scaffolding of claim 1, wherein said struts are between about 0.08 to about 0.12 mm thick.

7. The scaffolding of claim 1, wherein said material comprises a metal.

8. The scaffolding of claim 7, wherein said metal comprises at least one member selected from the group consisting of titanium, cobalt, chrome, tantalum, stainless steel, magnesium, and shape-memory alloys.

9. The scaffolding of claim 7, further comprising ceramic particles, whiskers or fibers on said metal.

10. The scaffolding of claim 1, wherein said material comprises a ceramic.

11. The scaffolding of claim 10, wherein said ceramic comprises at least one member selected from the group consisting of alumina, partially stabilized zirconia, hydroxyapatite, and calcium phosphates.

12. The scaffolding of claim 10, wherein said ceramic comprises hydroxyapatite doped with at least one member selected from the group consisting of Si, Mg, and carbonate.

13. The scaffolding of claim 1, wherein said material comprises a polymer.

14. The scaffolding of claim 13, wherein said polymer comprises at least one member selected from the group consisting of reinforced polymers, nylon, polycarbonate, polymethylmethacrylate, polyethylene, polyurethane, polyaryl etherketone, polyetheretherketone, polylactide, polyglycolide, and synthetic or natural collagen, poly(DL-lactide), poly(L-lactide), poly(glycolide), poly(ε-caprolactone), poly(dioxanone), poly(glyconate), poly(hydroxybutyrate), poly(hydroxyvalerate), poly(orthoesters), poly(carboxylates), poly(propylene fumarate), poly(phosphates), poly(carbonates), poly(anhydrides), poly(iminocarbonates), poly(phosphazenes), and copolymers, blends and combinations thereof.

15. The scaffolding of claim 13, wherein said polymer comprises at least one member selected from the group consisting of polyethylenes, high density polyethylene, ultra high molecular weight polyethylene, low density polyethylene, polybutylene, polystyrene, polyurethane, polypropylene, polyaryletherketone, polyacrylates, polymethacrylates, polymethylmethacrylate, polymerized monomers, tri(ethylene glycol) dimethacrylate, bisphenol a hydroxypropyl methacrylate, and copolymers, blends and combinations thereof.

16. The scaffolding of claim 13, wherein said polymer comprises an exothermic phase-change polymer.

17. The scaffolding of claim 16, wherein said bonded layers further comprise an evacuated layer to provide thermal protection.

18. The scaffolding of claim 1, wherein said material comprises a compressible or foldable material that may be expanded be internal pressurization.

19. The scaffolding of claim 1, wherein said material comprises at least two materials.

20. The scaffolding of claim 1, wherein said material is impregnated or coated with at least one of a drug and a medicine.

21. The scaffolding of claim 1, wherein said scaffolding comprises a fully open-celled structure.

22. The scaffolding of claim 1, wherein said scaffolding comprises a partially open-celled structure.

23. The scaffolding of claim 1, wherein said scaffolding comprises a fully closed-cell structure.

24. The scaffolding of claim 1, wherein said porosity of said material is not uniform among said layers.

25. The scaffolding of claim 1, wherein the density of said material is not uniform among said layers.

26. The scaffolding of claim 1, wherein the morphology of said material is not uniform among said layers.

27. The scaffolding of claim 1, wherein said material is coated with one member selected from the group consisting of diamond, aluminum oxide, ceramic, cermet, metal, metal alloy, polymer, biologic material, hydroxyapatite, and hyaluronic acid.

28. The scaffolding of claim 27, wherein said biologic material comprises animal tissue.

29. The scaffolding of claim 27, wherein said biologic material comprises vegetable matter.

30. The scaffolding of claim 27, wherein said biologic material comprises human tissue.

31. The scaffolding of claim 1, wherein said material comprises a transition in microstructure.

32. The scaffolding of claim 1, wherein said bonded layers comprise one or more materials, and said material comprises a transition between said one or more materials.

33. The scaffolding of claim 1, wherein said bonded layers further comprise a barrier layer.

34. The scaffolding of claim 1, wherein said material further comprises a solid layer on at least one of the top and bottom of said bonded layers to facilitate bonding to a solid structure.

35. The scaffolding of claim 1, wherein said bonded layers comprise a solid layer bonded to a metal reinforced polymer layer.

36. The scaffolding of claim 35, wherein said metal reinforced polymer layer is further bonded to a barrier layer.

37. The scaffolding of claim 36, wherein said barrier layer is further bonded to a high molecular weight acrylic polymer layer.

38. The scaffolding of claim 1, wherein said bonded layers further comprise a solid layer encompassing pressurized fluid.

39. The scaffolding of claim 38, wherein said solid layer comprises metal, composite, or a flexible polymer.

40. The scaffolding of claim 1, wherein the uppermost layer of said bonded layers is configured to integrate with bone and/or tissue.

41. The scaffolding of claim 40, wherein the layer disposed immediately below the bone and/or tissue integrating layer comprises a bioresorbable material layer.

42. The scaffolding of claim 41, wherein said bioresorbable material layer encapsulates at least one of a drug and a medicine.

43. The scaffolding of claim 41, wherein below said bioresorbable material layer is disposed a solid layer.

44. The scaffolding of claim 40, wherein the layer disposed immediately below the bone and/or tissue integrating layer comprises one or more layers transitioning to a fully dense layer in the region distal to the bone and/or tissue integrating layer.

45. The scaffolding of claim 44, wherein below said fully dense layer is disposed a transition layer bonded to an injection molded polymer.

46. The scaffolding of claim 44, wherein below said fully dense layer is disposed a second layer configured to integrate with bone and/or tissue.

47. The scaffolding of claim 1, wherein said material comprises a metal scaffold containing a liquid methylmethacrylate monomer.

48. The scaffolding of claim 47, wherein above said metal scaffold is bonded a barrier layer with an acrylic polymer layer further disposed thereon.

49. The scaffolding of claim 47, wherein said metal scaffold is bonded to a solid metal implant.

50. The scaffolding of claim 1, wherein said material comprises a piezoelectric material.

51. The scaffolding of claim 50, wherein said piezoelectric material comprises at least one member selected from the group consisting of quartz, barium titanate, rochelle salt, lead zirconium titanate (PZT), lead niobium oxide, and polyvinyl fluoride.

52. The scaffolding of claim 50, wherein said piezoelectric material is encapsulated by another material comprising a metal, polymer or ceramic.

53. A method for producing a bone and tissue in-growth scaffolding, comprising:
providing sheets of machined material, wherein said material comprises at least one of a metal, a ceramic and a polymer, wherein said material has a porosity between about 5% and about 95%, has cells with mean spacing between about 0.05 mm and about 5 mm, and has about 0.05 mm to about 2 mm thick struts;
subjecting said sheets to compression; and
bonding said sheets to produce a bone and tissue in-growth scaffolding.

54. The method of claim 53, wherein said sheets are produced in batches.

55. The method of claim 53, further comprising removing slag, splatter, maskant or contaminants from said sheets prior to stacking said sheets.

56. The method of claim 53, wherein said sheets are stacked prior to subjecting said sheets to compression.

57. The method of claim 56, wherein said sheets are stacked in a random manner.

58. The method of claim 56, wherein said sheets are stacked in an ordered fashion.

59. The method of claim 53, wherein said sheets are all bonded in one bonding step.

60. The method of claim 53, further comprising first producing said sheets of machined material by laser machining, chemical machining or etching, water jet cutting, electrical discharge machining, stamping, photochemical machining, plasma etching, electron beam machining or textile manufacturing processing.

61. The method of claim 53, further comprising first producing said sheets of material by machining slits in the material and expanding the slit material.

62. The method of claim 53, wherein said porosity is between about 50% and about 90%.

63. The method of claim 53, wherein said porosity is between about 70% and about 90%.

64. The method of claim 53, wherein said cells have a mean spacing of between about 0.25 mm and 0.6 mm.

65. The method of claim 53, wherein said struts are between about 0.08 to about 0.12 mm thick.

66. The method of claim 53, wherein said material comprises a metal.

67. The method of claim 66, wherein said metal comprises at least one member selected from the group consisting of titanium, cobalt, chrome, tantalum, stainless steel, magnesium, and shape-memory alloys.

68. The method of claim 53, wherein said material comprises a ceramic.

69. The method of claim 68, wherein said ceramic comprises at least one member selected from the group consisting of alumina, partially stabilized zirconia, hydroxyapatite, and calcium phosphates.

70. The method of claim 68, wherein said ceramic comprises hydroxyapatite doped with at least one member selected from the group consisting of Si, Mg, and carbonate.

71. The method of claim 53, wherein said sheets comprise individual layers of ceramic produced from a ceramic slurry.

72. The method of claim 53, wherein said material comprises a polymer.

73. The method of claim 72, wherein said polymer comprises at least one member selected from the group consisting of reinforced polymers, nylon, polycarbonate, polymethylmethacrylate, polyethylene, polyurethane, polyaryl etherketone, polyetheretherketone, polylactide, polyglycolide, and synthetic or natural collagen, poly(DL-lactide), poly(L-lactide), poly(glycolide), poly(ε-caprolactone), poly(dioxanone), poly(glyconate), poly(hydroxybutyrate), poly(hydroxyvalerate), poly(orthoesters), poly(carboxylates), poly(propylene fumarate), poly(phosphates), poly(carbonates), poly(anhydrides), poly(iminocarbonates), poly(phosphazenes), and copolymers, blends and combinations thereof.

74. The method of claim 72, wherein said polymer comprises at least one member selected from the group consisting of polyethylenes, high density polyethylene, ultra high molecular weight polyethylene, low density polyethylene, polybutylene, polystyrene, polyurethane, polypropylene, polyaryletherketone, polyacrylates, polymethacrylates, polymethylmethacrylate, polymerized monomers, tri(ethylene glycol) dimethacrylate, bisphenol a hydroxypropyl methacrylate, and copolymers, blends and combinations thereof.

75. The method of claim 53, wherein said material comprises at least two materials.

76. The method of claim 53, wherein said material comprises a coated second material.

77. The method of claim 53, wherein said material comprises a hybrid metal-ceramic scaffold.

78. The method of claim 53, wherein said scaffolding comprises a fully open-celled structure.

79. The method of claim 53, wherein said scaffolding comprises a partially open-celled structure.

80. The method of claim 53, wherein said scaffolding comprises a fully closed-cell structure.

81. The method of claim 53, wherein said sheets are stacked in a mold prior to subjecting said sheets to compression.

82. The method of claim 81, wherein said mold comprises a material having a higher melting temperature than the sheet material.

83. The method of claim 81, wherein said mold comprises a material that is chemically inert with respect to the sheet material.

84. The method of claim 81, wherein said mold comprises graphite.

85. The method of claim 81, wherein said mold is tightened to subject said sheets to compression.

86. The method of claim 81, further comprising heating said mold in a furnace to diffusion bond said sheets.

87. The method of claim 86, wherein said heating is conducted at a temperature that is roughly 90% of the melting point of said material on an absolute temperature scale.

88. The method of claim 53, wherein said sheets are bonded by one member selected from the group consisting of adhesive bonding, diffusion bonding, hot pressing, friction welding, ultrasonic welding, cold welding, laser welding, resistance welding, arc welding, brazing, and glazing.

89. The method of claim 53, wherein said sheets are from about 10 μm to about 1 mm in thickness.

90. The method of claim 53, wherein said bone and tissue scaffolding is approximately 2 mm to 3 mm in thickness.

91. The method of claim 53, wherein said sheets are wrapped around a mandrel prior to bonding said sheets.

92. The method of claim 91, wherein said mandrel comprises graphite.

93. The method of claim 53, wherein said bone and tissue in-growth scaffolding is designed using computer modeling to mimic the geometry of live tissue.

94. The product of the method of claim 53.
